SEQUENCE LISTING (2 sequences)

SEQ ID NO: 1 - DNA, 2408 bp, Ebola virus
cttgatgaagattaaggcaaccagtggtgctatcttcatctctttgatttgagtcttaagtgaatacacaggttctaatactgttcttctgtccaacggtataattcagccaggcctaagacagtagctaatcacagtcatcatgggagcgtcagggattctgcaattgccccgtgagcgcttcaggaaaacatctttctttgtttgggtaataatcctattccataaagtcttttcaatcccgttgggggttgtacacaacaataccctacaagtgagtgatattgacaagtttgtgtgccgagacaaactctcttcaactagccaattgaagtcagtcgggttgaacttggagggcaatggagtagcaactgatgtaccaacggcaaccaaaagatggggttttcgagctggtgttccaccaaaggtggtaaattacgaagctggagaatgggctgagaactgttataacctggctataaagaaagttgatggtagtgagtgcctaccagaagcccctgagggagtgagggattttccccgttgccgctatgtacacaaagtctcaggaactggaccatgcccaggaggactcgcctttcacaaagaaggagccttcttcctgtatgaccgactcgcatcaacaatcatttatcggggtacaacctttgccgaaggagttattgcatttctgatcttgcctaaggcgcgaaaggattttttccagtctcctccattgcatgagcctgccaacatgaccacggatccctccagttactatcacacgacaacaataaactacgtggttgataattttggaaccaacaccacagagtttctgttccaagtcgatcatttgacgtatgtgcagctcgaggcaagattcacaccacaattccttgtcctcctaaatgaaaccatctactctgataaccgcagaagtaacacaacaggaaaactaatctggaaaataaatcccactgttgataccagcatgggtgagtgggctttctgggaaaataaaaaaacttcacaaaaaccctttcaagtgaagagttgtctttcgtacctgtaccagaaacccagaaccaggtccttgacacgacagcgacggtctctcctcccatctccgcccacaaccacgcaggcgaagaccacaaagaattggtttcagaggattccactccagtggttcagatgcaaaacatcaagggaaaggacacaatgccaaccacagtgacgggtgtaccaacaaccacaccctctccatttccaatcaatgctcgcaacactgatcataccaaatcatttatcggcctggaggggccccaagaagaccacagcaccacacagcctgccaagaccaccagccaaccaaccaacagcacagaatcgacgacactaaacccaacatcagagccctccagtagaggcacgggaccatccagccccacggtccccaacaccacagaaagccacgccgaacttggcaagacaaccccaaccacactcccagaacagcacactgccgccagtgccattccaagagccgtgcaccccgacgaactcagtggacctggcttcctgacgaacacaatacggggggtgacaaatctcctgacaggatccagaagaaagcgaagggatgtcactcccaatacacaacccaaatgcaacccaaacctgcactattggacagccttggatgagggtgctgccataggtttagcctggataccatacttcgggccagcagctgagggaatttacactgaaggcataatggagaatcaaaatggattgatctgtggattgaggcagctggccaacgaaacgacacaagctcttcaattgttcttaagggcaactactgagttgcgtacattctctatactaaatcggaaagcaatagacttcttgctccaaagatggggaggaacatgtcacattctagggcctgattgttgcattgaaccccaagattggaccaaaaatatcactgataaaattgatcaaataatccatgactttgtcgataataatcttccaaatcagaatgatggcagcaactggtggactggatggaaacaatgggttcctgctggaataggaatcacaggagtaatcattgctattattgctttgctgtgcatttgcaaattcatgctttgaactaatatagcatcatactttctaatattcccccaatatgaattttgttttcgattttatttaatgatatatcctctgtacacctcactaatgtactcgagcataatttccctgatagacttgattgtatttgatgattaaggacctcacaaaattcctggggattgaaaagaactggataactcaataaattttatgctaggaccacaaatacacttgatgaagattaagaaaaa

SEQ ID NO: 2 - protein, 676 aa, Ebola virus
MGASGILQLPRERFRKTSFFVWVIILFHKVFSIPLGVVHNNTLQVSDIDKFVCRDKLSSTSQLKSVGLNLEGNGVATDVPTATKRWGFRAGVPPKVVNYEAGEWAENCYNLAIKKVDGSECLPEAPEGVRDFPRCRYVHKVSGTGPCPGGLAFHKEGAFFLYDRLASTIIYRGTTFAEGVIAFLILPKARKDFFQSPPLHEPANMTTDPSSYYHTTTINYVVDNFGTNTTEFLFQVDHLTYVQLEARFTPQFLVLLNETIYSDNRRSNTTGKLIWKINPTVDTSMGEWAFWENKKNFTKTLSSEELSFVPVPETQNQVLDTTATVSPPISAHNHAGEDHKELVSEDSTPVVQMQNIKGKDTMPTTVTGVPTTTPSPFPINARNTDHTKSFIGLEGPQEDHSTTQPAKTTSQPTNSTESTTLNPTSEPSSRGTGPSSPTVPNTTESHAELGKTTPTTLPEQHTAASAIPRAVHPDELSGPGFLTNTIRGVTNLLTGSRRKRRDVTPNTQPKCNPNLHYWTALDEGAAIGLAWIPYFGPAAEGIYTEGIMENQNGLICGLRQLANETTQALQLFLRATTELRTFSILNRKAIDFLLQRWGGTCHILGPDCCIEPQDWTKNITDKIDQIIHDFVDNNLPNQNDGSNWWTGWKQWVPAGIGITGVIIAIIALLCICKFML

(19) United States
(12) Patent Application Publication
(10) Pub. No.: US 2011/0217328 A1
(43) Pub. Date: Sep. 8, 2011

(54) COMPOSITIONS AND METHODS FOR TREATING EBOLA VIRUS INFECTION

(75) Inventor: Thomas William Geisbert, Boston, MA (US)

(73) Assignee: Trustees of Boston University, Boston, MA (US)

(21) Appl. No.: 12/809,370

(22) PCT Filed: Dec. 17, 2008
(86) PCT No.: PCT/US08/13798

§ 371 Date: Sep. 16, 2010

Related U.S. Application Data
(60) Provisional application No. 61/014,669, filed on Dec. 18, 2007.

(51) Int. Cl. A61K 39/12; C12N 15/63; A61P 31/14

(52) U.S. Cl. 424/199.1; 435/320.1

(57) ABSTRACT

The compositions and methods of the invention described herein provide treatments against Ebola virus infection by expressing gene(s) from the Ivory Coast ebolavirus (ICEBOV) species in a recombinant viral vector.

### BACKGROUND OF THE INVENTION

There are four distinct species of Ebola virus: Zaire ebolavirus (ZEBOV), Sudan ebolavirus (SEBOV), Ivory Coast ebolavirus (ICEBOV) (also known as Cote d'Ivoire ebolavirus (CIEBOV)), and Reston ebolavirus (REBOV). A new unnamed species of Ebola virus is suspected to be the causative agent of a recent outbreak of Ebola virus in Uganda. Three of these species (ZEBOV, SEBOV, and ICEBOV) and the newly identified species from Uganda cause fatal disease in humans. The developments of countermeasures against Ebola viruses have focused on SEBOV and ZEBOV, the two species that have historically been responsible for nearly all Ebola outbreaks. Studies have shown that vaccines based on the ZEBOV species are not able to protect nonhuman primates against SEBOV challenge, suggesting that an Ebola virus vaccine will likely require the inclusion of both ZEBOV and SEBOV proteins. Indeed, nonhuman primates that are immune to SEBOV are not protected against challenge with ZEBOV, while nonhuman primates that are immune to ZEBOV are not protected against SEBOV. Thus, there exists a need in the art for an effective Ebola virus vaccine that provides both pre- and post-exposure protection against all three pathogenic species of Ebola virus.

### SUMMARY OF THE INVENTION

The compositions and methods of the invention described herein provide treatments against Ebola virus infection by expressing one or more genes (e.g., two or more genes) from the Ivory Coast ebolavirus (ICEBOV) species in a subject in need thereof, which stimulates an immune response against the polypeptides encoded by the one or more genes, or by delivering a vehicle (e.g., a recombinant viral vector or a liposome) that includes one or more genes (e.g., two or more genes) or polypeptides from the Ivory Coast ebolavirus (ICEBOV) species to a subject in need thereof, which stimulates an immune response against the polypeptides present on the vehicle or the one or more genes delivered by the vehicle. In one embodiment, the pharmaceutical composition of the invention includes a recombinant viral vector that encodes at least one gene from the Ivory Coast species of Ebola virus. The pharmaceutical composition may further include a pharmaceutically acceptable diluent, excipient, carrier, or adjuvant. The viral vector (e.g., a recombinant, replication-defective vesicular stomatitis virus (rVSV) vector) may encode, e.g., all or part of the ICEBOV glycoprotein (SEQ ID NO: 1). The pharmaceutical composition may be, e.g., a vaccine. Preferably, the vaccine inhibits or treats infection by one or more of, e.g., ICEBOV, Zaire ebolavirus (ZEBOV), or Sudan ebolavirus (SEBOV), or any new strain or species of Ebola virus that may emerge such as the new Ebola virus from Uganda. The pharmaceutical composition may also alleviate, reduce the severity of, or reduce the occurrence of, one or more of the symptoms (e.g., fever, hemorrhagic fever, severe headache, muscle pain, malaise, extreme asthenia, conjunctivitis, popular rash, dysphagia, nausea, vomiting, bloody diarrhea followed by diffuse hemorrhages, delirium, shock, jaundice, thrombocytopenia, lymphocytopenia, neutrophilia, focal necrosis in various organs (e.g., kidneys and liver), and acute respiratory distress) associated with Ebola virus infection (e.g., infection by ICEBOV, ZEBOV, or SEBOV).

In another embodiment, the invention features a method of inhibiting or treating Ebola virus infection in a subject (e.g., a human) by administering to the subject a recombinant viral vector that encodes at least one gene from the Ivory Coast species of Ebola virus (e.g., the ICEBOV glycoprotein). The infection may be caused by the ICEBOV, ZEBOV, SEBOV, or any new strain or species of Ebola virus, such as the new Ebola virus from Uganda. The subject being treated may not have, but is at risk of developing, an infection by an Ebola virus. Alternatively, the subject may already be infected with an Ebola virus. The composition may be administered, e.g., by injection (e.g., intramuscular, intraarterial, intravascular, intravenous, intraperitoneal, or subcutaneous). The composition of the method may include, e.g., between 1×10¹ and 1×10⁸ pfu of the viral vector, preferably between 1×10² and 1×10⁸ pfu, more preferably between 1×10³ and 1×10⁸ pfu, or most preferably between 1×10⁴ and 1×10⁸ pfu. The composition may include, e.g., at least 1×10³ pfu of the viral vector (e.g., 1×10⁴ pfu of the viral vector). The method may include, e.g., administering the composition to the subject two or more times.

The invention also features a method of inducing an immune response to Ebola virus in a subject (e.g., a human) that includes administering to a subject a recombinant viral vector that encodes at least one gene from the Ivory Coast species of Ebola virus (e.g., the ICEBOV glycoprotein). The infection may be caused by the ICEBOV, ZEBOV, SEBOV, or any new strain or species of Ebola virus, such as the new Ebola virus from Uganda. The subject being treated may not have, but is at risk of developing, an infection by an Ebola virus. Alternatively, the subject may already be infected with an Ebola virus. The composition may be administered, e.g., by injection (e.g., intramuscular, intraarterial, intravascular, intravenous, intraperitoneal, or subcutaneous). The composition of the method may include, e.g., between 1×10¹ and 1×10⁸ pfu of the viral vector, preferably between 1×10² and 1×10⁸ pfu, more preferably between 1×10³ and 1×10⁸ pfu, or most preferably between 1×10⁴ and 1×10⁸ pfu. The composition may include, e.g., at least 1×10³ pfu of the viral vector (e.g., 1×10⁴ pfu of the viral vector). The method may include, e.g., administering the composition two or more times.

As used herein, by “administering” is meant a method of giving a dosage of a pharmaceutical composition of the invention to a subject. The compositions utilized in the methods described herein can be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, intraarterial, intravascular, and intramuscular administration. The preferred method of administration can vary depending on various factors (e.g., the components of the composition being administered and the severity of the condition being treated).

By “an amount sufficient to treat” is meant the amount of a composition administered to improve, inhibit, or ameliorate a condition of a subject, or a symptom of a disorder, in a clinically relevant manner (e.g., improve, inhibit, or ameliorate infection by Ebola virus or one or more symptoms that occur following infection by Ebola virus). Any improvement in the subject is considered sufficient to achieve treatment. Preferably, an amount sufficient to treat is an amount that prevents the occurrence or one or more symptoms of ebolavirus infection or is an amount that reduces the severity of, or the length of time during which a subject suffers from, one or more symptoms of ebolavirus infection (e.g., by at least 10%, 20%, or 30%, more preferably by at least 50%, 60%, or 70%, and most preferably by at least 80%, 90%, 95%, 99%, or more, relative to a control subject that is not treated with a composition of the invention). A sufficient amount of the pharmaceutical composition used to practice the methods described herein (e.g., the treatment of Ebola virus infection) varies depending upon the manner of administration and the age, body weight, and general health of the subject being treated. Ultimately, the prescribers or researchers will decide the appropriate amount and dosage.

As used herein, the term “gene” refers to a nucleic acid molecule that either directly or indirectly encodes a nucleic acid or protein product that has a defined biological activity.

By “ICEBOV glycoprotein” is meant the glycoprotein polypeptide, in secreted or transmembrane bound form, or any fragment or mutation of the glycoprotein polypeptide that is encoded by the ICEBOV genome (e.g., an ICEBOV polypeptide that includes amino acid residues 500-676 of SEQ ID NO: 2) so long as it has the ability to induce or enhance an immune response or confer a protective or therapeutic benefit to the subject, e.g., against one or more of the SEBOV, ZEBOV, ICEBOV, or a new strain or species of Ebola virus (e.g., the new Ebola virus from Uganda). An ICEBOV glycoprotein may also include any polypeptide, or fragment thereof, that is substantially identical (e.g., at least 60%, 65%, 70%, 75%, 80%, 85%, 90%, 95%, 98%, 99%, or even 100% identical) to the ICEBOV glycoprotein set forth in SEQ ID NO: 2 over at least 20, 25, 30, 35, 40, 45, 50, 55, 60, 65, or 70 contiguous residues.

By “inducing an immune response” is meant eliciting a humoral response (e.g., the production of antibodies) or a cellular response (e.g., the activation of T cells) directed against a virus (e.g., Ebola virus) in a subject to which the pharmaceutical composition (e.g., a vaccine) has been administered.

By “pharmaceutical composition” is meant any composition that contains at least one therapeutically or biologically active agent (e.g., at least one nucleic acid molecule or protein product, in whole or in part, of or corresponding to an ICEBOV genome, either incorporated into a viral vector or independent of a viral vector) and is suitable for administration to a subject. For the purposes of this invention, pharmaceutical compositions suitable for delivering a therapeutic or biologically active agent can include, e.g., tablets, gelcaps, capsules, pills, powders, granulates, suspensions, emulsions, solutions, gels, hydrogels, oral gels, pastes, eye drops, ointments, creams, plasters, drenches, delivery devices, suppositories, enemas, injectables, implants, sprays, or aerosols. Any of these formulations can be prepared by well-known and accepted methods of art. See, for example, Remington: The Science and Practice of Pharmacy (21^{St} ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

By “pharmaceutically acceptable diluent, excipient, carrier, or adjuvant” is meant a diluent, excipient, carrier, or adjuvant which is physiologically acceptable to a subject while retaining the therapeutic properties of the pharmaceutical composition with which it is administered. One exemplary pharmaceutically acceptable carrier is physiological saline. Other physiologically acceptable diluents, excipients, carriers, or adjuvants and their formulations are known to those skilled in the art.

By “recombinant,” with respect to a viral vector, is meant a vector (e.g., a viral genome that has been incorporated into one or more delivery vehicles (e.g., a plasmid, cosmid, etc.)) that has been manipulated in vitro, e.g., using recombinant nucleic acid techniques to introduce changes to the viral genome (e.g., to include heterologous viral nucleic acid sequences). An example of a recombinant viral vector of the invention is a vector that includes all or part of the VSV genome and that includes the nucleic acid sequence for all or part of the ICEBOV glycoprotein (see, e.g., U.S. Patent Application Publication No. 2006/0193872, hereby incorporated by reference).

By “subject” is meant any animal, e.g., a mammal (e.g., a human). A subject to be treated according to the methods described herein (e.g., a subject infected with, or at risk of being infected with, an Ebola virus) may be one who has been diagnosed by a medical practitioner as having such a condition. Diagnosis may be performed by any suitable means. A subject in whom the development of an infection is being prevented may or may not have received such a diagnosis. One skilled in the art will understand that a subject to be treated according to the present invention may have been identified using standard tests or may have been identified, without examination, as one at high risk due to the presence of one or more risk factors (e.g., exposure to Ebola virus, etc.).

By “treating” is meant administering a pharmaceutical composition for prophylactic and/or therapeutic purposes. Prophylactic treatment may be administered, for example, to a subject who is not yet ill, but who is susceptible to, or otherwise at risk of, a particular disorder, e.g., infection with an Ebola virus. Therapeutic treatment may be administered, for example, to a subject already suffering from a disorder in order to improve or stabilize the subject's condition (e.g., a patient already infected with an Ebola virus). Thus, in the claims and embodiments described herein, treating is the administration to a subject either for therapeutic or prophylactic purposes. In some instances, as compared with an equivalent untreated control, treatment may ameliorate a disorder or a symptom thereof by, e.g., 5%, 10%, 20%, 30%, 40%, 50%, 60%, 70%, 80%, 90%, 95%, or 100% as measured by any standard technique. In some instances, treating can result in the inhibition of viral infection, the treatment of the infection, and/or the amelioration of symptoms (e.g., hemorrhagic fever) of the infection. Confirmation of treatment can be assessed by detecting an improvement in or the absence of symptoms, or by the inability to detect the presence of Ebola virus in the treated subject.

By “viral vector” is meant a composition that includes one or more viral genes from two or more virus species that is able to transmit the genetic information to a host or subject. The nucleic acid material of the viral vector may be encapsulated, e.g., in a lipid membrane or by structural proteins (e.g., capsid proteins), that may include one or more viral polypeptides (e.g., an ICEBOV glycoprotein). The one or more viral genes of the viral vector may include, e.g., a nucleic acid (e.g., SEQ ID NO: 1; FIG. 1) that encodes one or more polypeptides (e.g., SEQ ID NO: 2; FIG. 2) of the ICEBOV species of Ebola virus. The viral vector can be used to infect cells of a subject, which, in turn, promotes the translation into a protein product of the one or more viral genes of the viral vector (e.g., an ICEBOV glycoprotein). The viral vector may also be, e.g., a pseudotyped virus that includes one or more of the polypeptides encoded by the genome of the Ivory Coast species of Ebola virus. The viral vector itself can be used to stimulate an immune response that is protective against infection by an Ebola virus (e.g., an ICEBOV) or that treats infection by an Ebola virus (e.g., an ICEBOV). Alternatively, the viral vector can be administered to a subject so that it infects one or more cells of the subject, which then promotes expression of the one or more viral genes of the viral vector and stimulates an immune response that is protective against infection by an Ebola virus (e.g., an ICEBOV) or that treats infection by an Ebola virus (e.g., an ICEBOV).

The term “vaccine,” as used herein, is a material used to provoke an immune response and confer immunity after administration of the material to a subject. For example, a vaccine of the invention may be a viral vector that includes one or more viral polypeptides (e.g., an ICEBOV glycoprotein) or that includes one or more viral genes that encode a viral polypeptide (e.g., an ICEBOV glycoprotein).

Other features and advantages of the invention will be apparent from the detailed description and from the claims.

### BRIEF DESCRIPTION OF THE DRAWINGS

FIG. 1 is the nucleotide sequence of the ICEBOV glycoprotein (SEQ ID NO: 1).

FIG. 2 is the polypeptide sequence of the ICEBOV glycoprotein (SEQ ID NO: 2).

FIG. 3 is the study design algorithm used to determine the minimal dose of a viral vector that expresses the ICEBOV glycoprotein that can be used as a preventive vaccine.

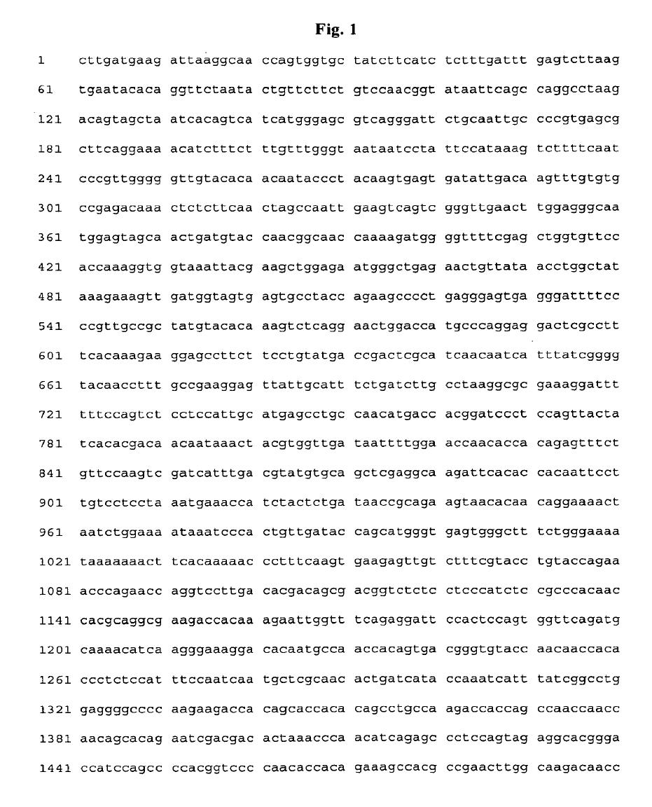

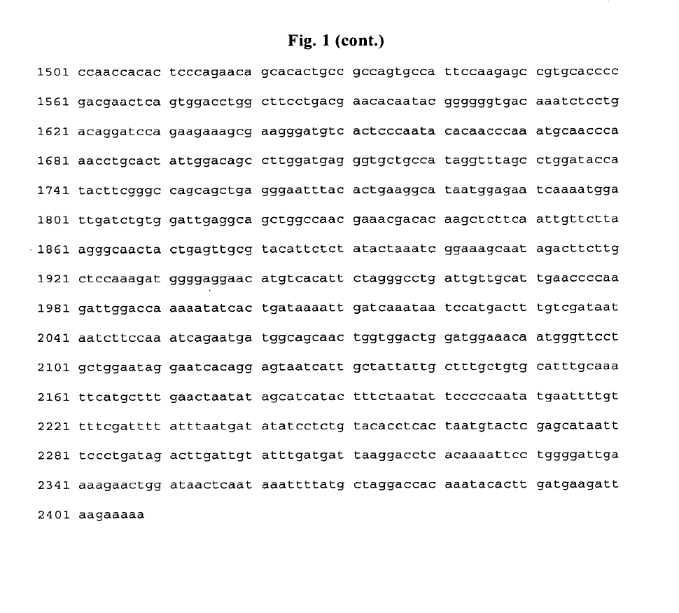

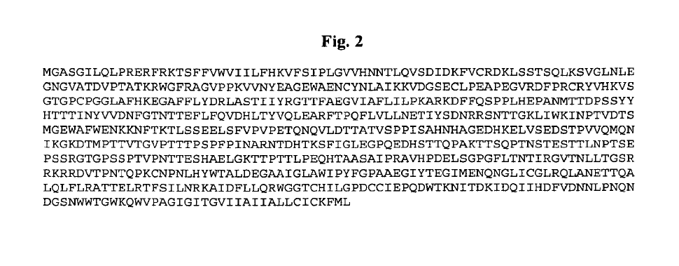

### DETAILED DESCRIPTION

The present invention features compositions and methods for treatment against Ebola virus infection by expressing one or more genes from the Ivory Coast ebolavirus (ICEBOV) species in a recombinant viral vector.

### Ebola Virus

Three species of Ebola virus are known to be pathogenic in humans: SEBOV, ZEBOV, and ICEBOV. A fourth species of Ebola virus is suspected to be the causative agent of a recent outbreak of Ebola virus in Uganda. The compositions and methods described herein utilize a gene or genes, or one or more polypeptides encoded by the gene or genes, from the ICEBOV species of Ebola virus to confer protection against all three pathogenic species of Ebola virus and the new species of Ebola virus from Uganda. The ICEBOV gene encoded in the viral vector of the invention may be, e.g., the ICEBOV glycoprotein, or a fragment thereof, that has the ability to induce or enhance an immune response that confers a protective or therapeutic benefit to the subject. The viral vector may also include an ICEBOV glycoprotein present on its surface. The ICEBOV glycoprotein, or the nucleic acid sequence encoding the ICEBOV glycoprotein, may have a mutation or deletion (e.g., an internal deletion, truncation of the amino- or carboxy-terminus, or a point mutation), so long as the mutation or deletion does not interfere with the immune response elicited by the glycoprotein following administration. The ICEBOV glycoprotein, or fragment thereof, which is present in a delivery vehicle of the present invention (e.g., a liposome or a viral vector), is capable of eliciting an immune response and may have, e.g., at least 5, 6, 7, 8, 9, 10, 20, 25, 30, 40, 50, 60, 70, 80, 90, 100, 125, 150, 175, 200, 300, 400, 500, 600, or more amino acid residues corresponding to the amino acid sequence set forth in SEQ ID NO: 2.

The ICEBOV glycoprotein may be obtained by any suitable means, including, e.g., application of genetic engineering techniques to a viral source, chemical synthesis techniques, recombinant production, or any combination thereof. The nucleic acid sequence of the ICEBOV glycoprotein (SEQ ID NO: 1) is published and is available from a variety of sources, including, e.g., GenBank and PubMed (e.g., GenBank No. U28006).

### Viral Vectors

In the invention described herein, a viral vector is utilized for the delivery of the pharmaceutical composition. Any suitable viral vector system can be used including, e.g., adenoviruses, adeno-associated viruses, rhabdoviruses (e.g., vesicular stomatitis virus), or poxviruses. The viral vector may be constructed using conventional techniques known to one of skill in the art. For example, the viral vector may contain at least one sequence encoding a heterologous gene (e.g., one or more genes) from the Ivory Coast species of Ebola virus (e.g., the glycoprotein (GP), secreted GP (sGP), major nucleocapsid protein (NP), RNA-dependent RNA polymerase (L), or one or more virion structural proteins (e.g., VP40, VP35, VP30, or VP24)), which is under the control of regulatory sequences that direct its expression in a cell. Alternatively, the viral vector is a pseudotyped virus that includes one or more of the polypeptides encoded by the genome of the Ivory Coast species of Ebola virus. Appropriate amounts for vector-mediated delivery of the ICEBOV gene(s) or for delivery of a pseudotyped virus can be readily determined by one of skill in the art, based on the information provided herein.

### Non-Viral Vectors

Non-viral approaches can also be employed for the introduction of therapeutic DNA or proteins into cells to treat or prevent Ebola virus infection. For example, an ICEBOV glycoprotein, or nucleic acid molecule encoding the same, can be introduced into a cell by lipofection (see, e.g., Feigner et al., Proc. Natl. Acad. Sci. USA 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or, less preferably, micro-injection under surgical conditions (Wolff et al., Science 247:1465, 1990). Gene transfer can also be achieved by the use of calcium phosphate, DEAE dextran, electroporation, and protoplast fusion. Liposomes, microparticles, or nanoparticles can also be potentially beneficial for delivery of DNA or protein (e.g., an ICEBOV glycoprotein) into a cell or into a patient in order to stimulate an immune response against the DNA or polypeptide.

### Therapy

Therapy according to the methods described herein may be performed alone or in conjunction with another therapy, and may be provided, e.g., at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the subject, the severity of the subject's infection, and how the subject responds to the treatment; the factors can be determined by one of skill in the art.

### Formulation and Administration of the Pharmaceutical Composition

The compositions utilized in the methods described herein can be administered by a route selected from, e.g., parenteral, intramuscular, intraarterial, intravascular, intravenous, intraperitoneal, subcutaneous, dermal, transdermal, ocular, inhalation, buccal, sublingual, perilingual, nasal, topical administration, and oral administration. The preferred method of administration can vary depending on various factors (e.g., the components of the composition being administered and the severity of the condition being treated). Formulations suitable for oral administration may consist of liquid solutions, such as an effective amount of the composition dissolved in a diluent (e.g., water, saline, or PEG-400), capsules, sachets or tablets, each containing a predetermined amount of the vaccine. The pharmaceutical composition may also be an aerosol formulation for inhalation, e.g., to the bronchial passageways. Aerosol formulations may be mixed with pressurized, pharmaceutically acceptable propellants (e.g., dichlorodifluoromethane, propane, or nitrogen).

Immunogenicity of the composition (e.g., vaccine) may be significantly improved if the composition of the present invention is co-administered with an immunostimulatory agent or adjuvant. Suitable adjuvants well-known to those skilled in the art include, e.g., aluminum phosphate, aluminum hydroxide, QS21, Quil A (and derivatives and components thereof), calcium phosphate, calcium hydroxide, zinc hydroxide, glycolipid analogs, octodecyl esters of an amino acid, muramyl dipeptides, polyphosphazene, lipoproteins, ISCOM matrix, DC-Chol, DDA, cytokines, and other adjuvants and derivatives thereof.

In some instances, it may be desirable to combine the compositions of the present invention with compositions that induce protective responses against other viruses. For example, the compositions of the present invention can be administered simultaneously, separately, or sequentially with other immunization vaccines, such as those for, e.g., influenza, malaria, tuberculosis, or any other vaccines known in the art.

Pharmaceutical compositions according to the invention described herein may be formulated to release the composition immediately upon administration (e.g., targeted delivery) or at any predetermined time period after administration using controlled or extended release formulations. Administration of the pharmaceutical composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window in the gastro-intestinal tract; or (iii) a short biological half-life, so that frequent dosing during a day is required in order to sustain a therapeutic level.

Many strategies can be pursued to obtain controlled or extended release in which the rate of release outweighs the rate of metabolism of the pharmaceutical composition. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

Administration of the pharmaceutical compositions (e.g., vaccines) of the present invention can be by any of the routes known to one of skill in the art. Administration may be by, e.g., intramuscular injection. The compositions utilized in the methods described herein can also be administered by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, and intramuscular administration. The preferred method of administration can vary depending on various factors, e.g., the components of the composition being administered and the severity of the condition being treated.

### Dosage

The pharmaceutical compositions of the invention are administered in such an amount as will be therapeutically effective, immunogenic, and/or protective against a pathogenic strain or species of Ebola virus. The dosage administered depends on the subject to be treated (e.g., the manner of administration and the age, body weight, capacity of the immune system, and general health of the subject being treated). The composition is administered in an amount to provide a sufficient level of expression that elicits an immune response without undue adverse physiological effects. Preferably, the composition of the invention is a heterologous viral vector that includes one or more polypeptides of the ICEBOV (e.g., the ICEBOV glycoprotein), or a nucleic acid molecule encoding one or more genes of the ICEBOV, and is administered at a dosage of, e.g., between 1×10¹ and 1×10⁸ pfu of the viral vector, preferably between 1×10² and 1×10⁸ pfu, more preferably between 1×10³ and 1×10⁸ pfu, or most preferably between 1×10⁴ and 1×10⁸ pfu. The composition may include, e.g., at least 1×10³ pfu of the viral vector (e.g., 1×10⁴ pfu of the viral vector). A physician or researcher can decide the appropriate amount and dosage regimen.

In addition, single or multiple administrations of the compositions of the present invention may be given to a subject. For example, subjects who are particularly susceptible to Ebola virus infection may require multiple treatments to establish and/or maintain protection against the virus. Levels of induced immunity provided by the pharmaceutical compositions described herein can be monitored by, e.g., measuring amounts of neutralizing secretory and serum antibodies. The dosages may then be adjusted or repeated as necessary to maintain desired levels of protection against viral infection.

### EXAMPLES

The present invention is illustrated by the following examples, which are in no way intended to be limiting of the invention.

### Example 1

### Construction of Recombinant VSV (rVSV) Expressing the ICEBOV Glycoprotein

The rVSV expressing the glycoprotein (GP) of ICEBOV is generated as described previously using the infectious clone for the VSV Indiana serotype (see, e.g., Garbutt et al., J. Virol. 78: 5458-65, 2004, and Jones et al., Nat Med 11: 786-90, 2005). Specifically, a plasmid containing five VSV genes (nucleoprotein (N), phosphoprotein (P), matrix protein (M), glycoprotein (G), and polymerase (L)), flanked by the bacteriophage T7 promoter sequence, the VSV leader sequence, the hepatitis virus delta virus ribozyme sequence, and the T7 terminator sequence is employed. Between the G and L genes, a unique linker site (Xho-NheI) is present, flanked by a transcriptional start and stop signal for an additional gene to be expressed. The open reading frame encoding the transmembrane glycoprotein of ICEBOV (e.g., GenBank No. U28006) is cloned into the XhoI and NheI sites of the modified full-length VSVXN2ΔG vector lacking the VSV G gene. The resulting plasmid is called pVSVXNΔG/ICEBOVGP. BSR-T7 cells are then grown to approximately 90% confluence in 6 cm dishes and the cells are transfected with the support plasmids encoding the viral ribonucleoprotein constituents (0.5 μg of pBS-VSV N, 1.25 μg of pBS-VSV P, and 0.25 μg of pBS-VSV L) and 2 μg of pVSVXNΔG/ICEBOVGP. Transfections are performed with Lipofectamine 2000 (Invitrogen) according to manufacturer's instructions. After 48 hours at 37° C., supernatants are blind passaged onto VeroE6 cells (80-90% confluent). Recovery of infectious virus is confirmed by surveying VeroE6 monolayers for cytopathic effects and by immunofluorescence assay tests (IFAT) and electron microscopy. Rescued recombinant VSVs are then passaged onto VeroE6 cells to obtain a virus vaccine stock. The vaccine vector is then titrated by plaque assay onto VeroE6 cells.

### Example 2

### Evaluation of the Protective Efficacy of VSVΔG/ICEBOVGP as a Preventive Vaccine Against ICEBOV, SEBOV, and ZEBOV in Cynomolgus Monkeys

A VSV vector of the invention expressing the ICEBOV GP (VSVΔG/ICEBOVGP) can be evaluated for its ability to protect animals against all three of the pathogenic Ebola virus species: ICEBOV, SEBOV, and ZEBOV. Because there are no rodent models of ICEBOV hemorrhagic fever (HF), these studies can be conducted in cynomolgus macaques. Previous efforts showed that ICEBOV caused severe clinical illness and viremia in 5 of 5 cynomolgus macaques (1000 pfu, intramuscular injection) and 3 of these 5 animals succumbed to the challenge.

Twelve filovirus-naïve cynomolgus monkeys are randomized into three experimental groups (Exp 1, Exp 2, and Exp 3) consisting of three monkeys each and three control groups (Cont 1, Cont 2, and Cont 3) consisting of one monkey each (Table 1). Animals in all three experimental groups receive approximately 2×10⁷ pfu of VSVΔG/ICEBOVGP. The control animals are injected in parallel with an equivalent dose of nonspecific vector (e.g., VSVΔG/LassaGPC). Animals in Exp 1 and Cont 1 are exposed to 1000 pfu of ICEBOV by intramuscular injection four weeks after the vaccination; animals in Exp 2 and Cont 2 are exposed to 1000 pfu SEBOV by intramuscular injection four weeks after the vaccination; and animals in Exp 3 and Cont 3 are exposed to 1000 pfu ZEBOV by intramuscular injection four weeks after the vaccination.

Animals are observed at least three times daily (at least 5 hours between each observation) during the entire study for evidence of clinical illness. Blood is collected 7 days before vaccination, immediately before vaccination, 2 and 14 days after vaccination, immediately before filovirus challenge and at days 3, 6, 10, 14, 21, and 28 after filovirus challenge. Physical exams, including weight and rectal temperature measurements, are performed each time the animals are anesthetized for blood collection. Clinical pathology evaluation includes a complete blood count (CBC) and a serum biochemistry panel (see, e.g., Daddario-DiCaprio et al., J Virol. 80:9659-9666, 2006). Levels of recombinant VSV and filoviruses in plasma are measured by viral infectivity titration and RT-PCR. Humoral immunity is assessed at each blood collection time point by IgG ELISA, while the cellular immune response is monitored by intracellular cytokine staining. Partial necropsies are performed on each animal at the study endpoint for gross pathological examination, and tissues (e.g., liver, spleen, lung, kidney, adrenal gland, axillary lymph nodes, inguinal lymph nodes, mesenteric lymph nodes, and brain) are collected and stored for virology and histology. For virology, tissues are stored at −70° C. For histology, tissues are fixed in 10% buffered formalin, processed, embedded in paraffin, and archived.

TABLE 1

| Group | Vaccine | Challenge virus | N |
|---|---|---|---|
| Exp 1 | VSVΔG/ICEBOVGP | ICEBOV | 3 |
| Exp 2 | VSVΔG/ICEBOVGP | SEBOV | 3 |
| Exp 3 | VSVΔG/ICEBOVGP | ZEBOV | 3 |
| Cont 1 | Control | ICEBOV | 1 |
| | (VSVΔG/LassaGPC) | | |
| Cont 2 | Control | SEBOV | 1 |
| | (VSVΔG/LassaGPC) | | |
| Cont 3 | Control | ZEBOV | 1 |
| | (VSVΔG/LassaGPC) | | |
| TOTAL | | | 12 |

### Example 3

### Evaluation of the Minimal Dose of VSVΔG/ICEBOVGP as a Preventive Vaccine Against Homologous ICEBOV in Cynomolgus Monkeys

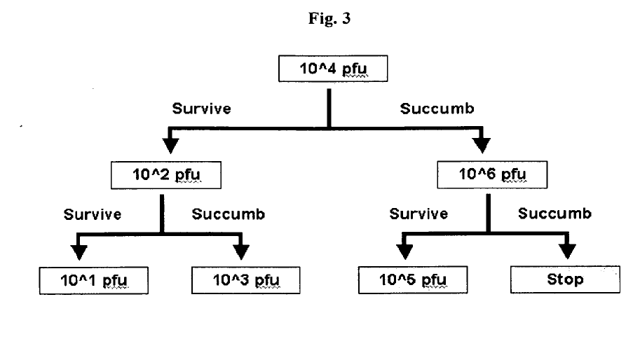

This study design follows the algorithm shown in FIG. 3. Briefly, three cynomolgus monkeys are vaccinated with a single injection of ˜1×10⁴ pfu of VSVΔG/ICEBOVGP and challenged 28 days later with 1000 pfu of homologous ICEBOV by intramuscular injection. A control animal is vaccinated in parallel with an equivalent dose of nonspecific rVSV vector (e.g., VSVΔG/LassaGPC) and challenged in parallel with ICEBOV. If all three animals vaccinated with VSVΔG/ICEBOVGP survive homologous ICEBOV challenge, the study is repeated using a lower vaccine dose, as shown in FIG. 3. If any of the three animals vaccinated with VSVΔG/ICEBOVGP succumb to homologous ICEBOV challenge, the study is repeated using a higher vaccine dose, as shown in FIG. 3. The study employs a minimum of 8 cynomolgus monkeys and a maximum of 12 cynomolgus monkeys.

### Example 4

### Evaluation of the Minimal Dose of VSVΔG/ICEBOVGP as a Preventive Vaccine Against Heterologous SEBOV in Cynomolgus Monkeys

The study design employed in Example 3 is repeated using the VSVΔG/ICEBOVGP for the vaccine against a heterologous challenge with SEBOV, as the minimal vaccine dose needed to confer protection against a homologous ICEBOV challenge may be different than the vaccine dose needed to confer protection against a heterologous SEBOV challenge. Briefly, three cynomolgus monkeys are vaccinated with a single injection of ˜1×10⁴ pfu of VSVΔG/ICEBOVGP and challenged 28 days later with 1000 pfu of heterologous SEBOV by intramuscular injection. A control animal is vaccinated in parallel with an equivalent dose of nonspecific rVSV vector (e.g., VSVΔG/LassaGPC) and challenged in parallel with SEBOV. If all three animals vaccinated with VSVΔG/ICEBOVGP survive the heterologous SEBOV challenge, the study is repeated using a lower vaccine dose. If any of the three animals vaccinated with VSVΔG/ICEBOVGP succumb to the heterologous SEBOV challenge, the study is repeated using a higher vaccine dose. The study employs a minimum of 8 cynomolgus monkeys and a maximum of 12 cynomolgus monkeys.

### Example 5

### Evaluation of the Minimal Dose of VSVΔG/ICEBOVGP as a Preventive Vaccine Against Heterologous ZEBOV in Cynomolgus Monkeys

The study design employed in Example 3 is repeated using the VSVΔG/ICEBOVGP for the vaccine against a heterologous challenge with ZEBOV, as the minimal vaccine dose needed to confer protection against a homologous ICEBOV challenge may be different than the vaccine dose needed to confer protection against a heterologous ZEBOV challenge. Briefly, three cynomolgus monkeys are vaccinated with a single injection of ˜1×10⁴ pfu of VSVΔG/ICEBOVGP and challenged 28 days later with 1000 pfu of heterologous ZEBOV by intramuscular injection. A control animal is vaccinated in parallel with an equivalent dose of nonspecific rVSV vector (e.g., VSVΔG/LassaGPC) and challenged in parallel with ZEBOV. If all three animals vaccinated with VSVΔG/ICEBOVGP survive the heterologous ZEBOV challenge, the study is repeated using a lower vaccine dose. If any of the three animals vaccinated with VSVΔG/ICEBOVGP succumb to the heterologous ZEBOV challenge, the study is repeated using a higher vaccine dose. The study employs a minimum of 8 cynomolgus monkeys and a maximum of 12 cynomolgus monkeys.

### Example 6

### Evaluation of Potential Interference Between VSVΔG/ICEBOVGP and VSVΔG/MARVGP-Musoke as a Preventive Vaccine Against ICEBOV, SEBOV, ZEBOV, MARV-Musoke, and MARV-Ravn

The objective of this experiment is to determine the interference between different component antigens in a multivalent filovirus vaccine. A multivalent preventive vaccine that can protect nonhuman primates against SEBOV, ZEBOV, ICEBOV, MARV-Ravn, and MARV-Musoke contains ICEBOV GP and MARV-Musoke GP. If ICEBOV is shown to protect nonhuman primates as a preventive vaccine against ICEBOV, SEBOV, and ZEBOV in studies described above (Example 1), Example 6 evaluates any interference between VSVΔG/ICEBOVGP and VSVΔG/MARVGP-Musoke when administered in equal parts as a blended vaccine. Example 6 is only performed if VSVΔG/ICEBOVGP protects nonhuman primates against ICEBOV, SEBOV, and ZEBOV as a preventive vaccine. If VSVΔG/ICEBOVGP does not protect nonhuman primates against ICEBOV, SEBOV, and ZEBOV as a preventive vaccine, Example 12 is performed instead of Example 7.

Twenty filovirus-naïve cynomolgus monkeys are randomized into five experimental groups (Exp 1, Exp 2, Exp 3, Exp 4, and Exp 5) consisting of three monkeys each and five control groups (Cont 1, Cont 2, Cont 3, Cont 4, and Cont 5) consisting of one monkey each. Animals in all five experimental groups receive an equal mixture of VSVΔG/MARVGP-Musoke and VSVΔG/ICEBOVGP (dose to be determined in dosing studies described above). Animals in the five control groups are injected in parallel with an equivalent total dose of nonspecific vectors (e.g., VSVΔG/LassaGPC). The study design is shown in Table 2, below. Animals in Exp 1 and Cont 1 are exposed to 1000 pfu ICEBOV by intramuscular injection four weeks after the vaccination; animals in Exp 2 and Cont 2 are exposed to 1000 pfu SEBOV by intramuscular injection four weeks after the vaccination; animals in Exp 3 and Cont 3 are exposed to 1000 pfu ZEBOV by intramuscular injection four weeks after the vaccination; animals in Exp 4 and Cont 4 are exposed to 1000 pfu MARV-Musoke by intramuscular injection four weeks after the vaccination; and animals in Exp 5 and Cont 5 are exposed to 1000 pfu MARV-Ravn by intramuscular injection four weeks after the vaccination. Animals are observed at least three times daily (at least 5 hours between each observation) during the entire study for evidence of clinical illness. Blood is collected 7 days before vaccination, immediately before vaccination, 2 and 14 days after vaccination, immediately before filovirus challenge and at days 3, 6, 10, 14, 21, and 28 after filovirus challenge. Physical exams, including weight and rectal temperature measurements, are performed each time animals are anesthetized for blood collection. Clinical pathology evaluation includes a complete blood count (CBC) and a serum biochemistry panel. Levels of recombinant VSV and filoviruses in plasma are measured by viral infectivity titration and RT-PCR. Humoral immunity is assessed at each blood collection time point by IgG ELISA, while the cellular immune response is monitored by intracellular cytokine staining. Partial necropsies are performed on each animal at the study endpoint for gross pathological examination, and tissues (liver, spleen, lung, kidney, adrenal gland, axillary lymph nodes, inguinal lymph nodes, mesenteric lymph nodes, and brain) are collected and stored for virology and histology. For virology, tissues are stored at −70° C. For histology, tissues are fixed in 10% buffered formalin, processed, embedded in paraffin, and archived.

TABLE 2

| Group | Vaccine | Challenge virus | N |
|---|---|---|---|
| Exp 1 | VSVΔG/ICEBOVGP + | ICEBOV | 3 |
| | VSVΔG/MARVGP-Musoke | | |
| Exp 2 | VSVΔG/ICEBOVGP + | SEBOV | 3 |
| | VSVΔG/MARVGP-Musoke | | |
| Exp 3 | VSVΔG/ICEBOVGP + | ZEBOV | 3 |
| | VSVΔG/MARVGP-Musoke | | |
| Exp 4 | VSVΔG/ICEBOVGP + | MARV-Musoke | 3 |
| | VSVΔG/MARVGP-Musoke | | |
| Exp 5 | VSVΔG/ICEBOVGP + | MARV-Ravn | 3 |
| | VSVΔG/MARVGP-Musoke | | |
| Cont 1 | Control (VSVΔG/LassaGPC) | ICEBOV | 1 |
| Cont 2 | Control (VSVΔG/LassaGPC) | SEBOV | 1 |
| Cont 3 | Control (VSVΔG/LassaGPC) | ZEBOV | 1 |
| Cont 4 | Control (VSVΔG/LassaGPC) | MARV-Musoke | 1 |
| Cont 5 | Control (VSVΔG/LassaGPC) | MARV-Ravn | 1 |
| TOTAL | | | 20 |

### Example 7

### Evaluation of the Protective Efficacy of VSVΔG/ICEBOVGP as a Post-Exposure Treatment Against SEBOV and ZEBOV in Rhesus Monkeys

As noted previously, the rVS V-based vaccines expressing filovirus GPs, when used as preventive vaccines, were shown to provide complete protection of nonhuman primates against homologous ZEBOV challenge (and heterologous MARV challenge). These rVSV-based filovirus vaccines also have utility as a post-exposure treatment for Marburg virus (Musoke strain) infection, ZEBOV infection, and SEBOV infection, in a manner similar to the post-exposure use of the rabies vaccine. However, most of the post-exposure treatment studies using rVSV vectors have treated animals with rVSV vectors based on the same strain or species of filovirus as the challenge virus (e.g., animals challenged with MARV-Musoke strain were treated with a VSVΔG/MARVGP-Musoke strain vector). As addressed herein, Example 1 focuses on the use of VSVΔG/ICEBOVGP as a preventive vaccine to protect nonhuman primates against ICEBOV, SEBOV, and ZEBOV. The protective efficacy of VSVΔG/ICEBOVGP as a post-exposure treatment against SEBOV and ZEBOV can be evaluated in rhesus monkeys, relative to previous SEBOV and ZEBOV post-exposure treatment studies in rhesus monkeys. Specifically, in previous studies, treatment of rhesus monkeys with homologous VSVΔG/ZEBOVGP vector 20-30 minutes after a high-dosage (1000 pfu) ZEBOV challenge resulted in protection of 4 of 8 animals from death. In comparison, treatment of rhesus monkeys with homologous VSVΔG/SEBOVGP vector 20-30 minutes after a high-dosage (1000 pfu) SEBOV challenge resulted in protection of 4 of 4 monkeys from death.

Ten filovirus-naïve rhesus monkeys are randomized into two experimental groups (Exp 1 and Exp 2) consisting of four monkeys each and two control groups (Cont 1 and Cont 2) consisting of one monkey each. The study design is shown in Table 4, below. Animals in Exp 1 and Cont 1 are exposed to 1000 pfu of SEBOV by intramuscular injection. Approximately 20 to 30 minutes after SEBOV challenge, animals in Exp 1 receive an intramuscular injection at four different sites of the VSVΔG/ICEBOVGP vector (˜2×10⁷ pfu) while the animal in Cont 1 receives an equivalent dose of a nonspecific vector (e.g., VSVΔG/LassaGPC). Animals in Exp 2 and Cont 2 are exposed to 1000 pfu of ZEBOV by intramuscular injection. Approximately 20 to 30 minutes after ZEBOV challenge, animals in Exp 2 receive an intramuscular injection (at four different sites) of the VSVΔG/ICEBOVGP vector (˜2×10⁷ pfu) while the animal in Cont 2 receives an equivalent dose of a nonspecific vector (e.g., VSVΔG/LassaGPC). Animals are observed at least three times daily (at least 5 hours between each observation) during the entire study for evidence of clinical illness. Blood is collected 7 days before vaccination, immediately before vaccination, 2 and 14 days after vaccination, immediately before filovirus challenge and at days 3, 6, 10, 14, 21, and 28 after filovirus challenge. Physical exams, including weight and rectal temperature measurements, are performed each time animals are anesthetized for blood collection. Clinical pathology evaluation includes a complete blood count (CBC) and a serum biochemistry panel. Levels of recombinant VSV and filoviruses in plasma are measured by viral infectivity titration and RT-PCR. Humoral immunity is assessed at each blood collection time point by IgG ELISA, while the cellular immune response is monitored by intracellular cytokine staining. Partial necropsies are performed on each animal at the study endpoint for gross pathological examination, and tissues (liver, spleen, lung, kidney, adrenal gland, axillary lymph nodes, inguinal lymph nodes, mesenteric lymph nodes, and brain) are collected and stored for virology and histology. For virology, tissues are stored at −70° C. For histology, tissues are fixed in 10% buffered formalin, processed, embedded in paraffin, and archived.

TABLE 4

| Group | Challenge virus | Post-exposure treatment | N |
|---|---|---|---|
| Exp 1 | SEBOV | VSVΔG/ICEBOVGP | 4 |
| Exp 2 | ZEBOV | VSVΔG/ICEBOVGP | 4 |
| Cont 1 | SEBOV | Control | 1 |
| | | (VSVΔG/LassaGPC) | |
| Cont 2 | ZEBOV | Control | 1 |
| | | (VSVΔG/LassaGPC) | |
| TOTAL | | | 10 |

### Other Embodiments

All publications and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

What is claimed is:

1. A pharmaceutical composition comprising a recombinant viral vector that encodes at least one gene from the Ivory Coast species of Ebola virus (ICEBOV).

2. The pharmaceutical composition of claim 1, further comprising a pharmaceutically acceptable diluent, excipient, carrier, or adjuvant.

3. The pharmaceutical composition of claim 1, wherein said viral vector encodes the ICEBOV glycoprotein gene or a fragment thereof.

4. The pharmaceutical composition of claim 3, wherein said ICEBOV glycoprotein comprises a sequence having at least 90% sequence identity to the sequence set forth in SEQ ID NO: 1.

5. The pharmaceutical composition of claim 1, wherein said recombinant viral vector is a vesicular stomatitis virus (rVSV) vector.

6. (canceled)

7. The pharmaceutical composition of claim 1, wherein said composition inhibits infection by ICEBOV, Zaire Ebola virus (ZEBOV), or Sudan Ebola Virus (SEBOV).

8. The pharmaceutical composition of claim 1, wherein said composition inhibits infection by all of ICEBOV, ZEBOV, and SEBOV.

9. The pharmaceutical composition of claim 1, wherein said composition alleviates the symptoms associated with Ebola virus infection.

10. (canceled)

11. The pharmaceutical composition of claim 9, wherein said Ebola virus is ICEBOV, ZEBOV, or SEBOV.

12. The pharmaceutical composition of claim 1, wherein said composition comprises between 1×10¹ and 1×10⁸ pfu of said viral vector.

13. (canceled)

14. A method of inhibiting or treating Ebola virus infection in a subject, said method comprising administering to said subject the composition of claim 1 in an amount sufficient to inhibit or treat said infection.

15. The method of claim 14, wherein said Ebola virus is Ivory Coast Ebola virus (ICEBOV), Zaire Ebola virus (ZEBOV), or Sudan Ebola Virus (SEBOV).

16. The method of claim 14, wherein said subject is not infected with said Ebola virus.

17. The method of claim 14, wherein said subject is infected with said Ebola virus.

18. (canceled)

19. (canceled)

20. The method of claim 14, wherein said composition comprises between 1×10¹ and 1×10⁸ pfu of said viral vector.

21. (canceled)

22. (canceled)

23. A method of inducing an immune response against Ebola virus infection in a subject, said method comprising administering to said subject the composition of claim 1 in an amount sufficient to inhibit or treat said infection.

24. The method of claim 23, wherein said Ebola virus is Ivory Coast Ebola virus (ICEBOV), Zaire Ebola virus (ZEBOV), or Sudan Ebola Virus (SEBOV).

25. The method of claim 23, wherein said subject is not infected with said Ebola virus.

26. The method of claim 23, wherein said subject is infected with said Ebola virus.

27. (canceled)

28. (canceled)

29. The method of claim 23, wherein said composition comprises between 1×10¹ and 1×10⁸ pfu of said viral vector.

30. (canceled)

31. (canceled)
